(19) United States
(12) Patent Application Publication
(10) Pub. No.: US 2002/0165365 A1
(43) Pub. Date: Nov. 7, 2002

(54) Synthetic catalyst for selective cleavage of protein and method for selective cleavage of protein using the same

(75) Inventors: Junghun Suh, Seoul (KR); Sang Jun Son, Seoul (KR); Jung Bae Song, Sungnam-shi (KR); Chang Eun Yoo, Seoul (KR); Chul-Seung Jeung, Seoul (KR); Joongwon Jeon, Seoul (KR); In Seok Hong, Seoul (KR)

Correspondence Address: Peter F. Corless, Dike, Bronstein, Roberts & Cushman, IP Group of, EDWARDS & ANGELL, LLP, 130 Water Street, Boston, MA, 02109, US

(73) Assignee: Artzyme Biotech Corporation, Seoul (KR)

(21) Appl. No.: 10/120,084

(22) Filed: Apr. 10, 2002

Related U.S. Application Data
(60) Provisional application No. 60/286,117, filed on Apr. 24, 2001.

(51) Int. Cl. C07K 16/46

(52) U.S. Cl. 530/391.1; 530/409; 540/474; 540/465; 546/12; 556/1; 562/565

(57) ABSTRACT

The present invention relates to a synthetic catalyst of the following formula (A) which can selectively recognize and cleave a specific protein among a protein mixture, and to a method for selective cleavage of a target protein using the same:

(R)(Z)ₙ (A)

in which

n denotes an integer of 1 or more,

R represents a material capable of selectively recognizing and binding a target protein, and

Z represents a metal ion-ligand complex.

### TECHNICAL FIELD

The present invention relates to a synthetic catalyst which can selectively recognize and cleave a specific protein among a protein mixture, and to a method for selective cleavage of a specific protein using the same. The selective cleavage of a specific protein makes it possible to selectively inhibit the biological activity of the protein.

### BACKGROUND ART

Proteins are responsible for a variety of biological functions in the living body. Particularly, since many enzymes and receptors are in charge of functions related to diseases, the molecules inhibiting those enzymes or receptors are frequently used as medicines. In case of enzymes, inhibitors reversibly block the active sites of enzymes to inhibit the enzyme function, whereas, in case of receptors, antagonists reversibly bind the receptors to reduce the receptor function (Medicinal Chemistry, 2nd Ed., Ganellin, C. R.; Roberts, S. M. Ed.; Academic Press: London, 1993). A suicide inhibitor is bound to the active site of the enzyme via a covalent bond to block the enzyme function. Many toxic proteins cause serious health problems as exemplified in prion for mad cow disease or amylod for Alzheimer's disease. For toxic proteins, synthetic molecules that specifically cleave the protein backbone may be used as effective medicines, but such molecules have not been reported.

When an inhibitor or an antagonist is added to an enzyme or a receptor, the degree of decrease in the concentration of protein having activity may be simply depicted as follows:

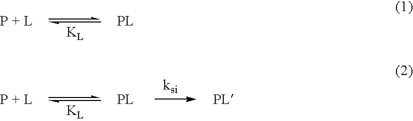

K_{L}=[P][L]/[PL] (3)

in which

P represents a protein having activity, and

L represents an inhibitor or an antagonist.

The scheme of Eq. (1) is related to a simple inhibitor or antagonist, and the scheme of Eq. (2) is related to a suicide inhibitor.

If L₅₀ represents the total concentration ([L]+[PL]+[PL′]) of L when the concentration of protein having activity ([P]) becomes half of the concentration of the total protein ([P]_{O}), L₅₀ is (KL+0.5[P]_{O}) in case of the scheme of Eq. (1). That is, L₅₀ decreases as K_{L} decreases but does not decrease to less than 0.5[P]_{O} when general inhibitors or antagonists are concerned. In case of the scheme of Eq. (2), L₅₀ is 0.5[P]_{O} and the time required for decreasing the concentration of L to a half level thereof shortens as K_{L} decreases or kₛᵢ increases. Inhibitors or antagonists having lower L₅₀ values are more effective as medicines. However, no matter how excellent inhibitors or antagonists may be, they cannot block biological activity of more than the equivalent amount of protein.

Some metal complexes are known to have the ability to cleave proteins. For example, the complexes formed between Cu(II) and cyclen, Cu(II) and 1,4,7-triazanonane, Cu(II) and tren, Pd(II) and ethylenediamine, and Fe(III) or Co(III) and coordinatively polymerized bilayer membranes are reported to be capable of hydrolyzing peptide bonds of proteins (Zhu, L.; Qin, L.; Parac, T. N.; Kostic, N. M. J Am. Chem. Soc. 1994, 116, 5218: Hegg, E. L.; Burstyn, J. N. J Am. Chem. Soc. 1995, 117, 7015: Suh, J.; Oh, S. Bioorg. Med. Chem. Lett. 1996, 6, 1067: Jang, B.-B.; Lee, K. P.; Min, D. H.; Suh, J. J. Am. Chem. Soc. 1998, 120, 12008: Moon, S.-J.; Jeon, J. W.; Kim, H.; Suh, M. P.; Suh, J. J. Am. Chem. Soc. 2000, 122, 7742: Suh, J.; Moon, S.-J. Inorg. Chem. 2001, 40, 4890). Further, it has been known that amides coordinated to Co(III) are hydrolyzed by Co(III) ion (Sutton, D. A.; Buckingham, D. A. Acc. Chem. Res. 1987, 20, 357). However, metal complexes that selectively attack and cleave a specific protein have never been reported up to date.

### DISCLOSURE OF INVENTION

As stated above, no matter how excellent inhibitors or antagonists may be, they cannot block biological activity of more than the equivalent amount of protein. In addition, synthetic catalysts specifically cleaving toxic proteins are not known. Therefore, the present inventors conducted extensive researches to overcome the fundamental limitation of the medicines acting as an inhibitor or an antagonist and to design synthetic catalysts specifically cleaving toxic proteins and, as a result, have designed a synthetic catalyst of the following formula (A):

(R)(Z)ₙ (A)

in which

n denotes an integer of 1 or more,

R represents a material capable of selectively recognizing and binding a target protein, particularly enzyme inhibitor or receptor antagonist, and

Z represents a metal ion-ligand complex.

Therefore, the purpose of the present invention is to provide a synthetic catalyst of formula (A), as defined above, which selectively binds and cleaves a target protein.

It is another purpose of the present invention to provide a method for selective cleavage of the target protein using the synthetic catalyst of formula (A).

### BEST MODE FOR CARRYING OUT THE INVENTION

Hereinafter, the synthetic catalyst of formula (A) according to the present invention is more specifically explained.

The synthetic catalyst of the present invention comprises group R as the site for recognition of a target protein, and this site can selectively bind the target protein to form a complex. After the recognition site is complexed to the target protein, the reaction site (Z) composed of a metal ion-ligand complex cleaves a peptide bond of the target protein. The protein thus cleaved is rapidly changed to a new conformation having a lower binding ability to the catalyst, and the catalyst is separated from the cleaved protein and regenerated to be used again to cleave other target protein molecules. Therefore, even if the binding ability of the synthetic catalyst to the target protein is not strong, a substantial amount of the target protein may be cleaved and the activity of the protein may be inhibited to a sufficient extent if sufficient time is allowed.

The mode of inhibiting activities of proteins by the synthetic catalyst according to the present invention may be simply represented by the following scheme which is similar to the Michaelis-Menten scheme:

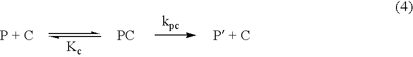

in which

P is defined as previously described,

C represents the synthetic catalyst of formula (A) according to the present invention,

P′ represents products obtained by the protein cleavage, and

K_{c} represents a constant corresponding to the Michaelis constant.

As can be seen from the scheme of Eq. (4), after the synthetic catalyst (C) is bound to the active protein (P) to form a complex (PC), it cleaves the protein to produce new proteins (P′) and at the same time regenerates itself. In this case, there is no limitation on the amount of catalyst required to inhibit the biological activity of the target protein (P) by cleaving a half thereof. The longer time for inhibiting the activity of the target protein to a specific level is allowed, the lower amount of catalyst may be used. As the synthetic catalyst forms a stronger complex (PC) with the target protein, K_{c} decreases, and as K_{c} decreases or k_{pc} increases, the rate for the protein cleavage increases.

As the recognition site R of the synthetic catalyst according to the present invention, any materials that can selectively recognize and bind the target protein may be used. An existing structure may be selected from the data accumulated in the past for the target protein, or otherwise a new structure may be designed.

When a target protein is an enzyme, any known inhibitors blocking the activity of the protein may be used. When a target protein is a receptor, any known antagonists binding to the receptor may be used. That is, if any information on the inhibitors or antagonists for a target protein is available, the existing inhibitors or antagonists may be used as the recognition site for preparing the custom-made synthetic catalyst for cleaving the protein. However, the position on the target protein, which the synthetic catalyst from the present invention binds to, may differ from those to which the existing inhibitors or antagonists bind. Inhibitors or antagonists bind to the sites that are essential to the activity of the target protein, whereas the synthetic catalyst according to the present invention may specifically recognize and bind the target protein at any positions including the active site. It is because the desired purpose of the present invention can be achieved simply by cleaving any peptide bond adjacent to the binding site. Therefore, a new structure having no relation with the existing inhibitors or antagonists may be used as the recognition site. Further, when the target protein is the one for which any inhibitors or antagonists are not reported, a new recognition site can be designed through the screenings using the synthetic r.; 1 catalyst of the present invention.

Depending on which target protein is selected, or on which inhibitor or antagonist is selected among those known for the target protein, or on whether a recognition site is newly designed or not, the recognition site R in the synthetic catalyst of the present invention may vary without limit. Thus, it is impossible to define the recognition site in the structural aspect.

In the structure of the above formula (A), the catalyst core corresponding to the reaction site Z is a metal complex such as the Cu(II) complex of cyclen. Possible metal complexes include those which cannot cleave protein or exhibit only scarcely detectable cleaving activity when they are unbound to the recognition site. The molecule synthesized by combining the metal complex with the protein recognition site, i.e. the synthetic catalyst, is complexed to the target protein to form a conjugate. In the conjugate of target protein and synthetic catalyst, the effective concentration between the metal complex and the cleavage site of the target protein can be sufficiently high to allow the effective cleavage of the peptide bond of the target protein.

The present inventors have discovered that, in achieving the purpose of inhibiting the biological activity of the target protein through a selective cleavage thereof using the synthetic catalyst as above, it is important to limit the kinds of metal ion and ligand constituting the complex to specific ones.

A variety of metal complexes having the ability of cleaving proteins have been known. The metal ions which can be suitably used as the constituent of the metal ion-ligand complex in the present invention comprise one or more selected from the group consisting of Ni(II), Cu(II), Zn(II), Pd(II), Cr(III), Fe(III), Co(III), Rh(III), Ir(III), Ru(III), Pt(IV), Zr(IV), and Hf(IV), preferably one or more selected from the group consisting of Cu(II), Cr(III), Fe(III), Co(III), Rh(III), Ir(III), and Ru(III). The skeleton of chelating ligands includes one or more selected from the group consisting of the following formula:

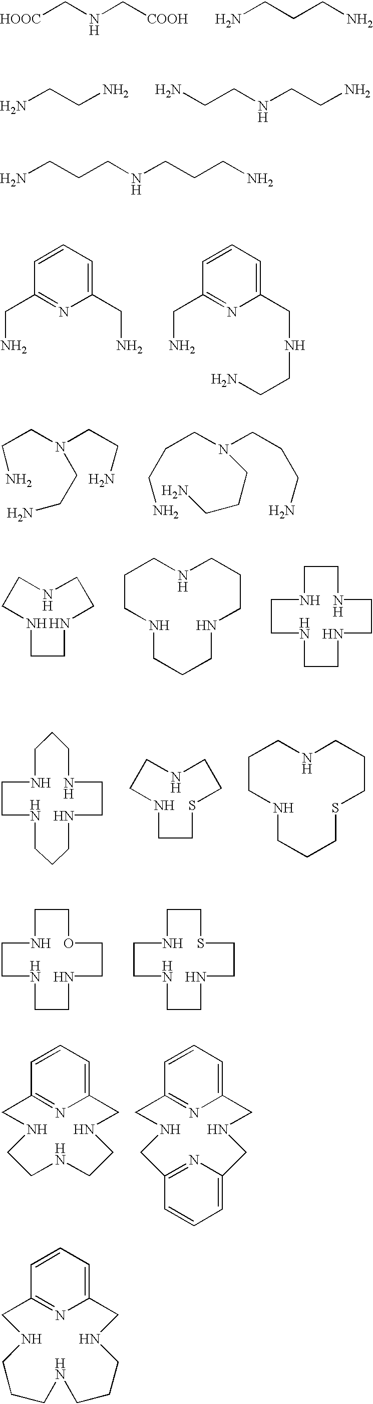

As can be seen from the above formulae, the chelating ligand according to the present invention is characterized in that it is cyclic or acyclic and one to four atoms among the metal-coordinating atoms contained in the ligand are nitrogen atoms. These nitrogen atoms may be either aromatic or non-aromatic nitrogen atoms.

In the structure of formula (A) according to the present invention, R and Z may be linked through a linker having a main chain directly connecting R with Z and optionally some side chains which are attached to the main chain. When the recognition site R is bound to a target protein, the reaction site Z cleaves one or more of the peptide bonds in the target protein. If the effective concentration between the cleavage site on the protein and the reaction site Z is increased, the reactivity of the reaction site Z may be improved. The efficient method for controlling the effective concentration is to control the relative positions between the recognition site (R) and the reaction site (Z) in the synthetic catalyst. The means for controlling the relative positions are lengths and shapes of linkers.

The linker should contain a main chain. The backbone of the main chain may be made of 1 to 30 atoms of boron, carbon, nitrogen, oxygen, silicon, phosphorus, and/or sulfur, which belong to functional groups such as alkyl, aryl, carbonyl, amine, ether, hydroxy, silyl, sulfhydryl, and/or thioether groups as well as derivatives such as amides, imides, esters, and/or thioesters. The linker may contain side chains, each of which has a backbone made of 1 to 30 atoms of boron, carbon, nitrogen, oxygen, silicon, phosphorus, and/or sulfur, belonging to functional groups such as alkyl, aryl, carbonyl, amine, ether, hydroxy, silyl, sulfhydryl, and/or thioether groups as well as derivatives such as acids, amides, imides, esters, and/or thioesters. Within the definition as explained above, the structures of linkers suitable for controlling the effective concentration between the cleavage site and the reaction site for the various target proteins and synthetic catalysts may be designed.

The reaction site (Z) in the synthetic catalyst according to the present invention can be combined with the recognition site (R) in a ratio of one or more reaction site(s) per one recognition site. The reaction sites may be identical with or different from each other. When one to three reaction sites are combined with respect to one recognition site, the examples of typical connection modes can be represented as follows. Otherwise, it is possible to insert the reaction site inside the recognition site.

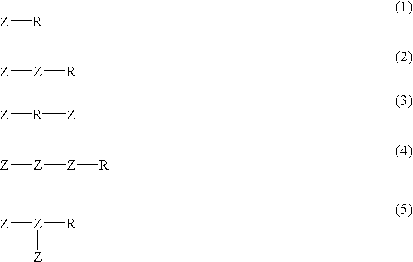

The present invention will be more specifically illustrated by the following examples. While the following examples are provided for the purpose of illustrating the present invention, they are not intended to be construed as limiting the scope of the present invention. Myoglobin (Mb) and avidin are used as the target proteins in the examples. For Mb, the catalysts are equipped with the recognition site discovered by using a newly prepared combinatorial library. For avidin, on the other hand, biotin is used as the recognition site of the catalyst, since biotin is known to strongly bind avidin. Various organic compounds are exploited in the examples as the chelating ligands of the reactive metal centers. In the examples, synthetic catalysts are added in molar amounts either greater or smaller than those of the target proteins.

### EXAMPLES

### Example 1

In search of the binding site of a protein-cleaving catalyst, we constructed a combinatorial library (CycAc(Q)ₙLysNH₂:Q is PNA monomer A′, G, T′, or C) of cyclen (Cyc) derivatives containing peptide nucleic acid (PNA) analogues. PNA analogues contain nucleobase analogues (NB(A′), NB(G), NB(T′), NB(C)) that can be used for base-pairing with nucleobases of DNA. NB(A′) and NB(T′) recognize NB(T)and NB(A), respectively. NB(A′) and NB(T′), however, do not recognize each other (Lohse, J.; Dahl, O.; Nielson, P. E. Proc. Natl. Acad Sci. U.S.A. 1999, 96, 10804). Base-pairing among PNA mixtures present in the library, therefore, can be suppressed by using A′ and T′ instead of A and T as the constituents of the PNAs.

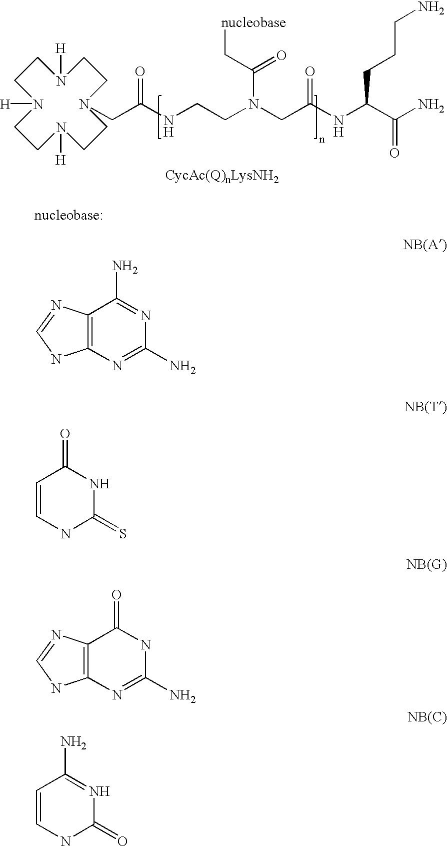

Fmoc derivative of A′ (N-[(2-amino-6-{[(benzyloxy)carbonyl]amino}-9H-purin9-yl)acetyl]-N-(2-{[(9H-fluoren-9-ylmethoxy)carbonyl]amino}ethyl)glycine (1)) was synthesized according to Scheme 1. To the stirred solution of (2-amino-6-{[(benzyloxy)carbonyl]amino}-9H-purin-9-yl)acetic acid (la) (2.0 g, 5.8 mmol) (Haaima, G.; Hansen, H.; Christensen, L.; Dahl, O.; Nielsen, P. Nucleic Acid Res. 1997, 25, 4639) in DMF (100 mL) were added the HCl salt of tert-butyl N-(2-{[(9H-fluoren9-ylmethoxy)carbonyl]amino}ethyl)glycinate (1b) (2.8 g, 6.4 mmol) (Thomson, S.; Josey, J.; Cadilla, R.; Gaul, M.; Hassman, C.; Luzzio, M.; Pipe, A.; Reed, K.; Ricca, D.; Wiethe, R.; Noble, S.; Tetrahedron 1995, 51, 6179) and triethylamine (TEA) (1.6 mL, 12 mmol). To the reaction mixture was added O-benzotriazol-1-yl-N,N,N′,N′-tetramethyluronium-hexafluorophosphate (HBTU) (2.4 g, 6.4 mmol), and the mixture was stirred for 3 h. The solution was evaporated and the residue was dissolved in methylene chloride (MC) (100 mL) The MC solution was washed with 5% aq. citric acid (50 mL×2), 5% aq. Na₂CO₃ (50 mL×2), and brine (50 mL×2), and dried over Na₂SO₄. The solvent was evaporated off, and flash chromatography afforded tert-butyl N-[(2-amino-6-{[(benzyl-oxy)carbonyl] amino} -9H-purin-9-yl)acetyl]-N-(2-{[(9H-fluoren-9-ylmethoxy)carbonyl]-amino}ethyl)glycinate (1c) as a white solid. R_{f} 0.4 (CH₃OH/MC 1:20); ¹H NMR (300 MHz, CDCl₃): δ 8.01 (S, 1H), 7.73 (m, 2H), 7.64 (m, 2H), 7.37-7.25 (m, 9H), 6.33 (s, 1H), 5.16 (s, 2H), 5.05 and 4.90 (s, 2H), 4.36 (m, 1H), 4.26 (m, 2H), 4.00 and 3.94 (s, 2H), 3.50 (s, 1H), 3.37(m, 2H), 3.15(s, 1H), 1.38(m, 9H). To the solution of 1c (1.5 g, 2.1 mmol) in MC (25 mL) was added trifluoroacetic acid (TFA) (25 mL). The reaction mixture was stirred for 5 h. After the solvent was evaporated off, flash chromatography afforded 1 as a white solid. R_{f} 0.3 (CH₃OH/MC 1:9); ¹H NMR (300 MHz, DMSO-d₆): δ 10.1 (s, 1H), 7.88 (m, 2H), 7.72 (s, 1H), 7.67 (m, 2H), 7.45-7.29 (m, 9H), 6.33 (s, 1H), 5.16 (s, 2H), 5.05 and 4.90 (s, 2H), 4.36 (m, 1H), 4.26 (m, 2H) 4.00 and 3.94 (s, 2H), 3.50 (s, 1H), 3.37 (m, 2H), 3.15 (s, 1HRMS exact mass 665.6874 (M+H)⁺ calcd for C₃₄H₃₃N₈O₇ 665.6854.

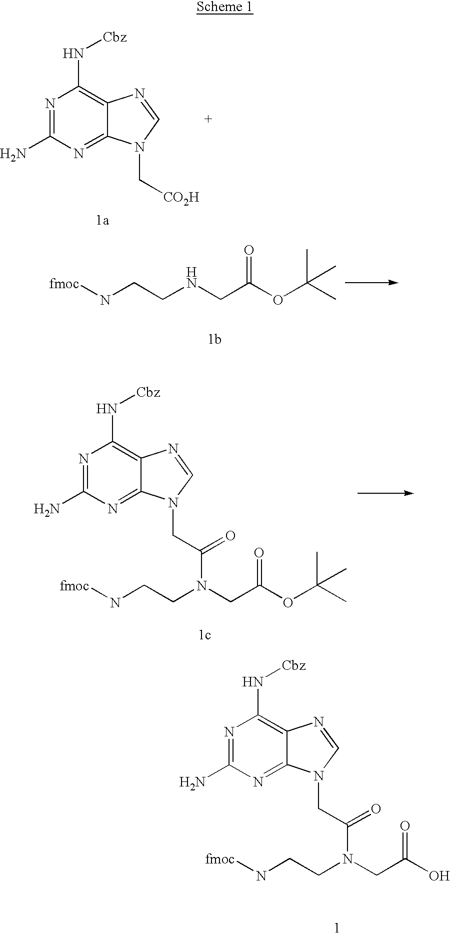

Fmoc derivative of T′ (N-{[2-(benzylthio)-4-oxopyrimidin-1(4H)-yl]acetyl}-N-(2-{[(9H-fluoren-9-ylmethoxy)carbonyl]amino}ethyl)glycine (2)) was synthesized according to Scheme 2. [2-(Benzylthio)-4-oxopyrimidin-1(4H)-yl]acetic acid (2a) was synthesized according to the literature (Lohse, J.; Dahl, O.; Nilesen, P. Proc. Natl. Acad. Sci. USA 1999, 96, 11804), except that benzyl group was used as the S-protecting group instead of methoxybenzyl group. ¹H NMR (300 MHz, DMSO-d6): δ 13.54 (br s 1H), 7.69 (d, 1H), 7.42 (d, 2H), 7.38-7.25 (m, 3H), 5.91 (d, 1H), 4.68 (s, 2H), 4.56 (s, 2H). To the stirred solution of 2a (3.6 g, 5.8 mmol) in DMF (100 mL) were added 1b (6.2 g, 6.4 mmol) and TEA (3.6 mL, 12 mmol). To the reaction mixture was added HBTU (5.4 g, 6.4 mmol) and the mixture was stirred for 3 h. The solvent was evaporated off and the residue was dissolved in MC (100 mL). The solution was washed with 5% aq. citric acid (50 mL×2), 5% aq. Na₂CO₃ (50 mL×2), brine (50 mL×2), and dried over Na₂SO₄. The solvent was evaporated off, and flash chromatography afforded tert-butyl N-{[2-(benzylthio)4-oxopyrimidin-1 (4-H)-yl]acetyl}-N-(2-{[(9H-fluoren-9-ylmethoxy)carbonyl]amino}ethyl)-glycinate (2b) as a white solid. R_{f} 0.4 (CH₃OH/MC 1:20); ¹H NMR (300 MHz, DMSO-d6): δ 7.87 (d, 2H), 7.64 (m, 2H), 7.47-7.20 (m, 10H), 5.90 (s, 1H), 4.96 (s, 1H), 4.72 (s, 1H), 4.35 (d, 2H), 4.27 (d, 2H), 4.18-4.16 (m, 2H), 4.14 (s, 1H), 3.36 (m, 2H), 3.30 (m, 1H), 3.28 (m, 1H), 1.38 (m, 9H). To the solution of 2b (3.0 g, 4.6 mmol) in MC (25 mL) was added TFA (25 mL). The reaction mixture was stirred for 5 h. After the solvent was evaporated off, flash chromatography afforded 2 as a white solid. R_{f} 0.3 (CH₃OH/MC 1:9); ¹H NMR (300 MHz, DMSO-d6): δ 7.87 (d, 2H), 7.64 (m, 2H), 7.39-7.20 (m, 10H), 5.90 (s, 1H), 4.96 (s, 1H), 4.76 (s, 1H), 4.35 (m, 1H), 4.23-4.16 (m, 3H), 4.14 (s, 1H), 3.39-3.35 (m, 2H), 3.32 (m, 1H), 3.15 (m, 1H); HRMS exact mass 599.6873 (M+H)⁺ , calcd for C₃₂H₃₂N₄O₆S 599.6875.

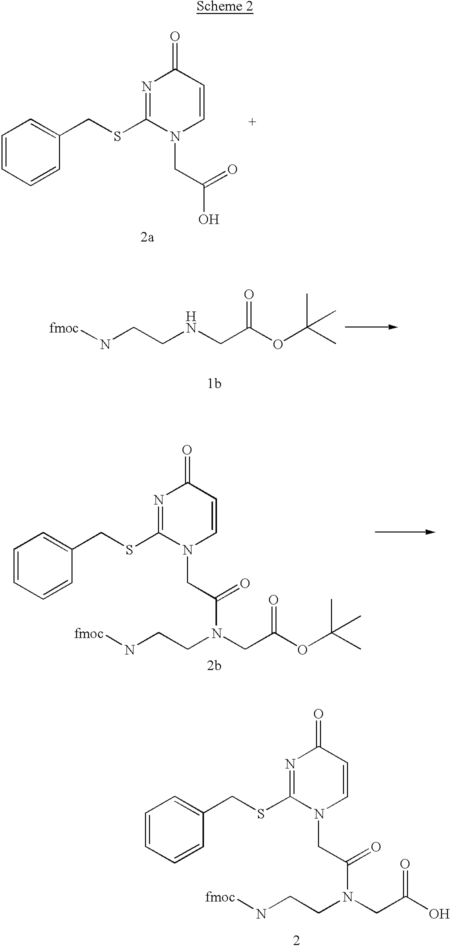

[4,7,10-Tris (tert-butoxycarbonyl)-1,4,7,10-tetraazacyclododecan-1-yl]acetic acid (3) was synthesized according to Scheme 3. To the mixture of tri-tert-butyl 1,4,7,10-tetraazacyclododecane-1,4,7-tricarboxylate (3a) (10 g, 21 mmol) (Kimura, E.; Aoki, S.; Koike, T.; Shiro, M. J Am. Chem. Soc. 1997, 119, 3068), Na₂CO₃ (2.5 g, 23 mmol), and CH₃CN (200 mL) was added ethyl bromoacetate (3.2 mL, 23 mmol). The heterogeneous mixture was refluxed overnight. After filtration, the solvent was evaporated off, and flash chromatography afforded tri-tert-butyl 10-(2-ethoxy-2-oxoethyl) 1,4,7, 10-tetraazacyclododecane-1,4,7-tricarboxylate (3b) as an amorphous solid. R_{f} 0.5 (ethyl acetate(EtOAc)/hexane 1:1); ¹H NMR (300 MHz, CDCl₃): δ 4.16 (q, 2H), 3.55-3.32 (br, 14B), 2.94 (br s, 4H), 1.45 (m, 27H), 1.32 (t, 3H). To the solution of 3b (10 g, 18 mmol) in CH₃OH (100 mL) was added aq. NaOH (1 N, 100 mL), and the reaction mixture was stirred for 2 h. The solvent was evaporated off, the residue was dissolved in 10% aq. citric acid, and pH was adjusted to 5. After the solution was extracted with EtOAc (100 mL×2) and the organic layer was dried over Na₂SO₄ and evaporated, 3 was obtained as an amorphous solid. ¹H NMR (300 MHz, CDCl₃): δ 3.53-3.30 (br, 14H), 2.96 (br s, 4H), 1.43 (m, 27H); MS (MALDI-TOF) m/z 531.75 (M+H)⁺ (C₂₅H₄₇N₄O₈ calcd. 531.67).

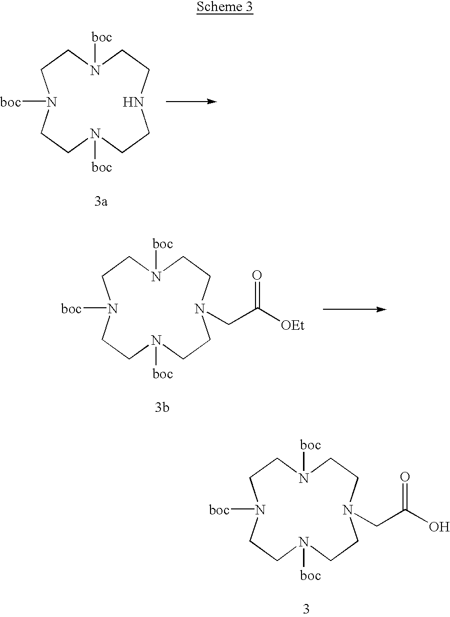

PNA monomers G and C protected with the fmoc group were purchased from Applied Biosystems and L-lysine protected with the fmoc group from Nova Biochem. PNAs for the combinatorial library (CycAc(Q)ₙLysNH₂) were synthesized by automated synthetic procedures using an Expedite Model 8909 Nucleic Acid Synthesis System with the fmoc-derivatives of A′, T′, G, C, and L-lysine as well as carboxylic acid 3. In synthesis of the library, it was assumed that the fmoc derivatives of A′, T′, G, and C are equally reactive in coupling with the growing PNA chain attached to the polymer support. Purity of PNA was confirmed by MALDI-TOF MS analysis using a Voyager-DE™ STR Biospectrometry Workstation model. The library of CycAc(Q)ₙLysNH₂ (total concentration: ca. 7×10⁻⁵ M) was mixed with an aqueous solution of CuCl₂ (3.5×10⁻⁴ M) to generate the library of Cu(II)CycAc(Q)ₙLysNH₂ where Cu(II) is bound to the Cyc moiety. A protein (ca. 1×10⁻⁵M) solution was added to this mixture to test cleavage of the protein. With the Cu(II)Cyc library containing up to 8 PNA monomers in each molecule, no evidence was obtained for cleavage of proteins such as bovine serum albumin, γ-globulin, elongation factor P, gelatin A, gelatin B, and horse heart Mb at 37° C. and pH 7.0 (50 mM 4-(2-hydroxyethyl)piperazine-1-ethanesulfonic acid (Hepes)) when checked by electrophoresis (SDS-PAGE). The Cu(II)Cyc library containing 9-mer PNAs in each molecule clearly showed activity for cleavage of Mb. We synthesized four groups of the library with the known PNA monomer positioned next to Cu(II)Cyc, and tested their activity for Mb cleavage to identify the best terminal monomer. By repeating the search for the rest of monomers, we chose Cu(II) complex of I (MS (MALDI-TOF) m/z 2851.49 (M+H)⁺ (C₁₁₁H₁₅₃N₆₄O₂₅S₂ calcd. 2850.58) as the best catalyst.

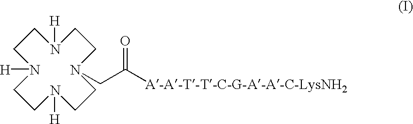

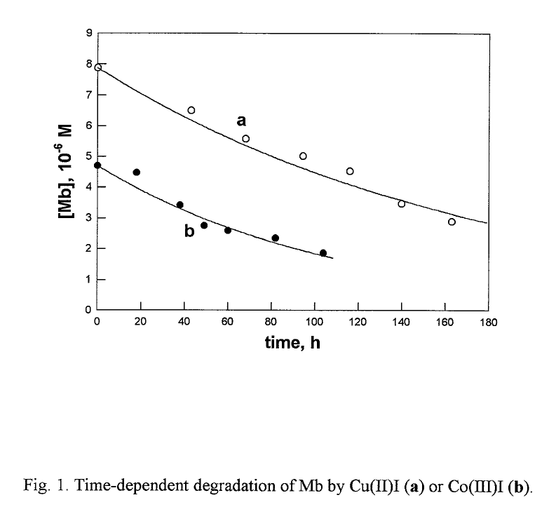

The stock solution of Cu(II) complex of I was prepared by adding an aqueous solution of CuCl₂ to I (1.2 equiv) dissolved in a buffer solution (1 mM 2-morpholinoethanesulfonic acid (Mes), pH 6.0). The degradation of Mb by Cu(II)I was followed by electrophoresis (SDS-PAGE). An example is illustrated in FIG. 1. Here, the reaction was carried out at pH 7.5 (50 mM Hepes) with [Mb]_{O} (the initially added concentration of Mb) of 7.9 μM and [Cu(II)I]₀ (the initially added concentration of Cu(II)I) of 2.0 μM. In 170 h, 2.5 molecules of Mb were degraded by each Cu(II)I molecule. The time-dependent decrease in [Mb] was fitted to pseudo-first-order kinetic equations, which produced the pseudo-first-order rate constant (k_{O}) of 5.7×10⁻³h⁻¹. The curves shown in FIG. 1 are obtained by fitting the data to pseudo-first-order kinetic equations. Removal of oxygen from the reaction mixture did not affect k_{O} appreciably. When Mb was treated with Cu(II)Cyc instead of Cu(II)I under the conditions otherwise identical to the above-mentioned experiment, degradation of Mb was not appreciable.

The Co(III) complex of I was obtained by incorporating Co(III) ion to the Cyc moiety of I according to the general method reported in the literature (Castillo-Blum, S. E.; Sosa-Torres, M. E. Polyhedron, 1995, 14, 223): for Co(III)I, MS (MALDI-TOF) m/z 2908.44 (M+H)⁺ (C₁₁₁H₁₅₃N₆₄O₂₅S₂Co calcd. 2908.51).

The degradation of Mb by Co(III)I was also followed by electrophoresis (SDS-PAGE). An example is illustrated in FIG. 1. Here, the reaction was carried out at pH 7.5 (50 mM Hepes) with [Mb]_{O} of 4.7 μM and [Co(III)I]_{O} of 0.47 μM. In 100 h, 6.0 molecules of Mb were degraded by each Co(III)I molecule. The time-dependent decrease in [Mb] was fitted to pseudo-first-order kinetic equations, which produced k_{O} of 9.4×10⁻³ h⁻¹. Removal of oxygen from the reaction mixture did not affect k_{O} appreciably. When Mb was treated with Co(III)Cyc instead of Co(III)I under the conditions otherwise identical to the above-mentioned experiment, degradation of Mb was not appreciable.

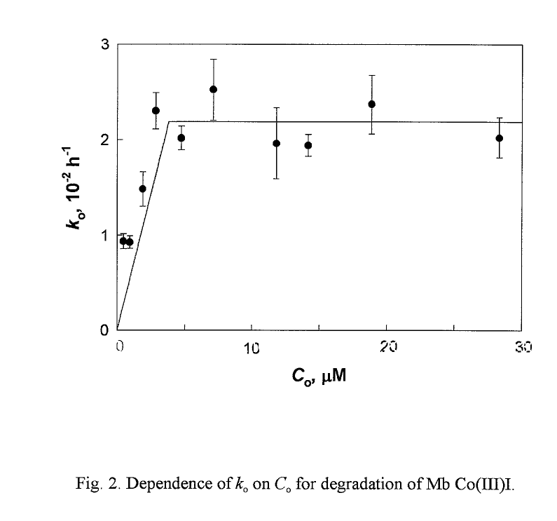

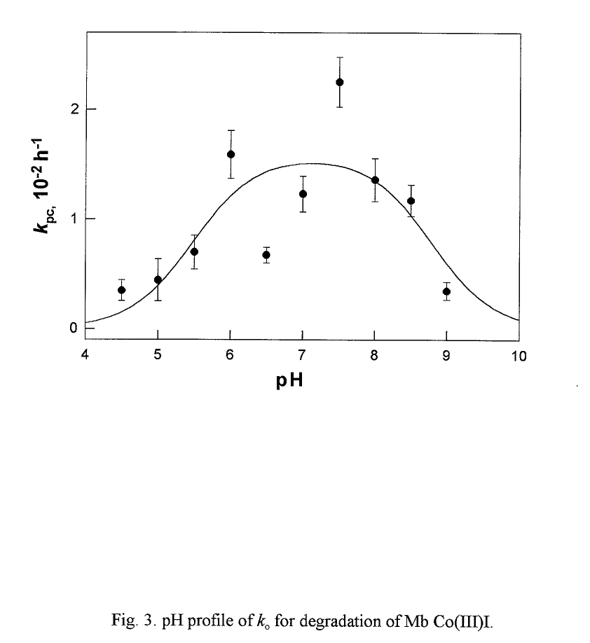

Although the structure of I was searched by using the Cu(II) complex, detailed kinetic analysis was performed with the Co(III) complex due to the higher catalytic activity of the Co(III) complex. The dependence of k₀ on C_{O} (the initially added concentration of the catalyst) measured at pH 7.5 is illustrated in FIG. 2. Here, [Mb]_{O} was fixed at 4.7 μM. The two straight lines drawn in FIG. 2 intersect at C_{O}=[Mb]₀. The kinetic data of FIG. 2 indicate that Mb is fully bound to Co(III)I when C_{O}≧[Mb]_{O}, and thus, K_{c}<<5 μM. Furthermore, k_{O} measured with C_{O} greater than [Mb]_{O} corresponds to k_{pc}, where K_{c} and k_{pc} are defined in Eq. (4). The k_{pc} values thus measured at various pHs are illustrated in FIG. 3 Analysis of the bell-shaped pH profile according to the scheme of Eq. (5) led to pKₐ₁=5.50+0.42 and pKₐ₂=8.68±0.46. The curve drawn in FIG. 3 is constructed on the basis of these pK values. If ionization of Mb or I is disregarded, these pKₐ values may be assigned to the ionization of aquo ligands of Co(III) ion of Co(III)I complexed to Mb.

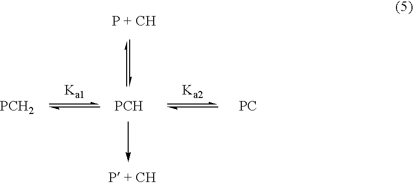

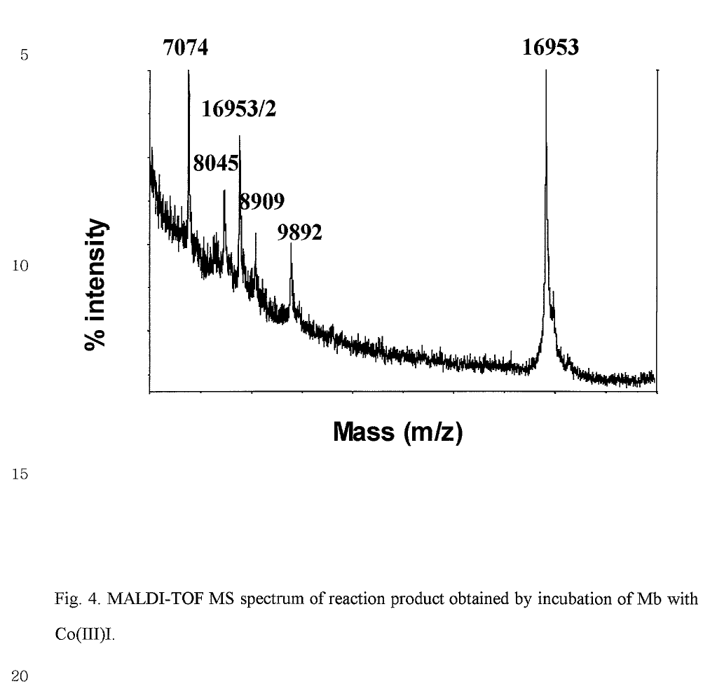

MALDI-TOF MS of the reaction mixture obtained by incubation of Mb (12 μM) with Co(III)I (3.5 μM) at pH 6.0 and 37° C. for 85 h disclosed that Mb was dissected into two pairs of proteins (M. W.: 7074 and 9892 for one pair and 8045 and 8909 for the other pair) as illustrated in FIG. 4. In FIG. 4, the peaks with m/z value 16953 and 16953/2 are due to Mb (M. W. 16953). Possible sites of the protein cleavage by Co(III)I are: Leu89-Ala90 (producing fragments with M. W. 7077 and 9894) and Leu72-Gly73 (producing fragments with M. W. 8057 and 8914) for the two pairs, respectively. It is not clear at present whether the two cleavage sites involve different binding modes of the catalyst. It is also possible that two cleavage sites originate from the same complex formed between Mb and the catalyst.

When other proteins such as bovine serum albumin, γ-globulin, elongation factor P, gelatin A, and gelatin B were incubated with Cu(II)I or Co(III)I, protein cleavage was not observed. This demonstrates that Cu(II)I or Co(III)I is specific for Mb.

An analogue of Co(III)I was prepared where the PNA residue next to the CycAc unit is C instead of A′ as indicated by Ia. No catalytic activity was observed for Co(III)Ia in the cleavage of Mb, indicating that Mb recognizes Co(III)I specifically.

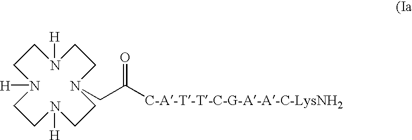

Up to 2.5 or 6 molecules of Mb were cleaved by each molecule of Cu(II)I or Co(III)I, respectively, in the data of FIG. 1, indicating the catalytic nature of the actions of Cu(II)I and Co(III)I. The reaction rate was unaffected by the removal of O₂ from the reaction mixtures. These results in combination with previous observations for hydrolytic cleavage of peptide bonds by Cu(II) complex of tetraaza ligands and Co(III) complexes (Moon, S.-J.; Jeon, J. W.; Kim, H.; Suh, M. P.; Suh, J. J Am. Chem. Soc. 2000, 122, 7742: Suh, J; Moon, S.-J. Inorg. Chem. 2001, 40, 4890: Sutton, D. A.; Buckingham, D. A. Acc. Chem. Res. 1987, 20, 357) support the hydrolytic nature of cleavage of Mb by Cu(II)I and Co(III)I.

### Example 2

Compound II was synthesized according to the method described in Example 1.

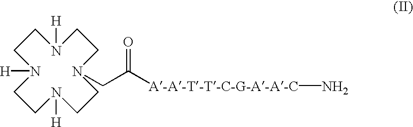

The Co(III) complex of II was obtained as described in Example 1. When Mb (12 μM) was incubated with Co(III)II (12 μM) at pH 7.0 or pH 8.0 (50 mM Hepes) and 37° C., Mb was degraded with k₀ of 1.4×10⁻²h⁻¹ or 6.9×10⁻³ h⁻¹ respectively. The results of Example 2 indicate that Lys of I is not required for the catalytic activity.

### Example 3

N²,N⁶-Bis {[4,7,10-tris(tert-butoxycarbonyl)-1,4,7,10-tetraazacyclododecan-1-yl]-acetyl}lysine (4) was synthesized according to Scheme 4. To the solution of bromoacetic acid (3.5 g, 26 mmol) in chloroform (100 mL) was slowly added N,N′-dicyclohexylcarbodiimide (5.3 g, 26 mmol). HCl salt of 4a (2 g, 8.58 mmol) was dissolved in chloroform (50 mL) completely by adding diisopropylethylamine (DIEA) (3.0 mL, 17 mmol) and this solution was slowly added to the solution of bromoacetic acid. After stirring for 8 h at room temperature, N,N′-dicyclohexylurea (DCU) was filtered off and the filtrate was evaporated. The residue was redissolved in CH₃CN (100 mL), and the undissolved DCU was filtered off. The filtrate was evaporated and flash chromatography afforded methyl N²,N⁶-bis(bromoacetyl)lysinate (4b) as a white solid. R_{f} 0.7 (EtOAc); ′H NMR (300 MHz, CDCl₃): δ 7.30 (br s, 1H), 6.71 (br s, 1H), 4.55 (m, 1H), 4.05 (d, 0.7H), 3.90 (m, 3.4H), 3.86 (s, 3H), 3.30 (m, 2H), 1.90 (m, 1H), 1.76 (m, 1H), 1.57 (m, 2H), 1.37 (m, 2H). To the mixture of 3a (3.1 g, 6.5 mmol), Na₂CO₃ (2.2 g, 19 mmol), and CH₃CN (100 mL) was added 4b (1.3 g, 3.2 mmol). The mixture was stirred and refluxed for 2 days. After filtration, the solvent was evaporated off, and flash chromatography afforded methyl N²,N⁶-bis{[4,7,10-tris(tert-butoxycarbonyl)-1,4,7,10-tetraazacyclododecan-1-yl]acetyl}ly-sinate (4c) as an amorphous solid. R_{f} 0.4 (CH₃OH/MC 1:40); ¹H NMR (300 MHz, CDCl₃): δ 7.06 (br s, 1H), 6.92 (br s, 1H), 4.50 (m, 1H), 3.71 (s, 3H), 3.13-3.53 (br m, 30H), 2.79-2.63 (br m, 8H), 1.84-1.65 (m, 4H), 1.44-1.47 (m, 54H), 1.36 (m, 2H). To the solution of 4c (1.7g, 1.4 mmol) in CH₃OH (50 mL) was added aq. NaOH (1 N, 50 mL) and the reaction mixture was stirred for 1 h. The solvent was evaporated off, the residue was dissolved in 10% aq. citric acid, and pH was adjusted to 4. After the solution was extracted with EtOAc (50 mL×2) and the organic layer was dried over Na₂SO₄ and evaporated, 4 was obtained as an amorphous solid. ¹H NMR (300 MHz, CD₃OD): 4.14 (m, 1H), 3.17-3.46 (br m, 28H), 3.14 (t, 2H), 2.79-2.70 (br m, 8H), 1.73 (m, 1H), 1.55 (m, 1H), 1.36 (m, 54H), 1.33-1.20 (m, 4H); MS (MALDI-TOF) m/z 1172.48 (M+H)⁺ (C₅₆H₁₀₃N₁₀O₁₆ calcd. 1172.49).

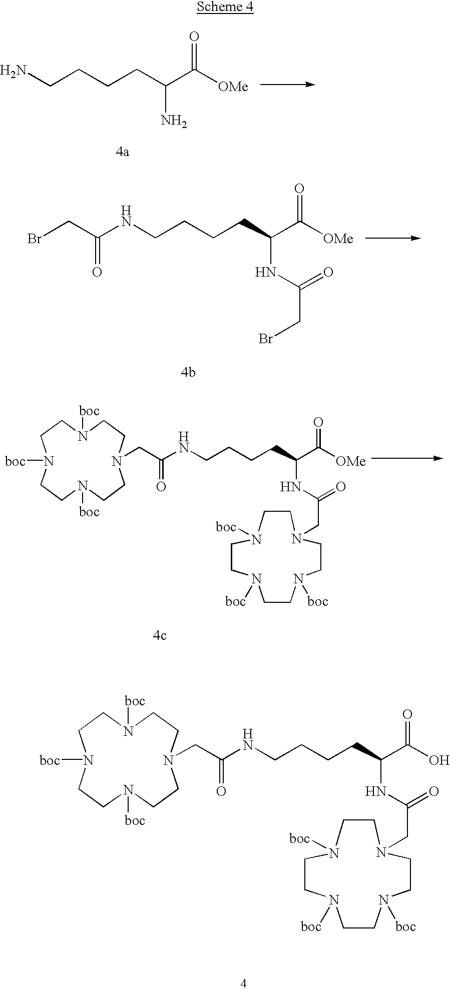

Compound III was synthesized by using 4 according to the method described in Example 1: MS (MALDI-TOF) m/z 3191.87 (M+H)⁺(C₁₂₇H₁₈₅N₇₀O₂₈S₂ calcd. 3190.23)

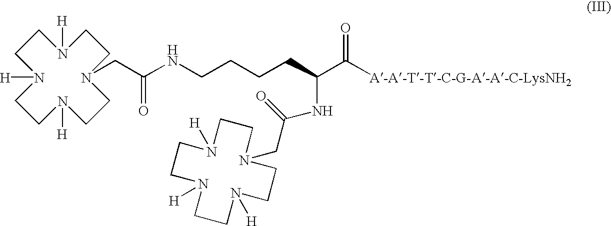

Complexation of Cu(II) ion to III and kinetic measurement for degradation of Mb were carried out as described in Example 1. When Mb (7.9 μM) was incubated with Cu(II)III (6.4 μM) at pH 8.0 (50 mM Hepes) and 37° C., Mb was degraded with k₀ of 3.3×10⁻³h⁻¹.

The Co(III) complex of III was obtained as described in Example 1. When Mb (7.9 μM) was incubated with Co(III)III (4.8 μM)) at pH 8.0 (50 mM Hepes) and 37° C., Mb was degraded with k_{O} of 3.2×10⁻³h⁻¹.

### Example 4

N-({ 4,7,1 0-Tris[(benzyloxy)carbonyl]-1,4,7,10-tetraazacyclododecan-1-yl}acetyl)-glycyl-N-(2-{[(9H-fluoren-9-ylmethoxy)carbonyl]amino}ethyl)glycine (5) was synthesized according to Scheme 5. Compound 5a ({4,7,10-tris[(benzyloxy)carbonyl]1,4,7,10-tetraazacyclododecan-1-yl}acetic acid) was synthesized by the procedure used for synthesis of 3, except that benzyl chloroformate was used instead of di-tert-butyl dicarbonate as the N-protecting group. ¹H NM (300 MHz, CDCl₃): δ 7.34 (m, 15H), 5.15 (m, 6H) 3.53-3.30 (br, 14H), 2.96 (br s, 4H). To the stirred solution of 5a (2 g, 3.2 mmol) in CH₃CN (100 mL) were added glycine ethyl ester hydrochloride (0.53 g, 3.8 mmol) and DIEA (1.4 mL, 7.9 mmol). To the reaction mixture was added HBTU (1.3 g. 3.5 mmol) and the mixture was stirred for 2 h. The solution was evaporated and the resulting residue was dissolved in EtOAc (100 mL). The solution was washed with 5% aq. citric acid (50 mL×2), 5% aq. Na₂CO₃ (50 mL×2), and brine (50 mL×2), and dried over Na₂SO₄. After filtering, the solvent was evaporated off, and flash chromatography afforded ethyl N-({4,7,10-tris[(benzyloxy)carbonyl]-1,4,7,10-tetraazacyclododecan-1-yl} acetyl)glycinate (5b) as an amorphous solid. R_{f} 0.5 (CH₃OH/MC 1:19); ¹H NMR (300 MHz, CDCl₃): 67 7.29 (m, 15H), 7.00 (s, 1H), 5.28 (s, 6H), 4.17-4.10 (m, 2H), 3.90 (br s, 2H), 3.40-3.15 (br m, 14H), 2.80 (br s, 4H), 1.26-1.22 (m,3H). To the solution of 5b (2.0 g, 2.8 mmol) in CH₃OH (50 mL) was added aq. NaOH (1 N, 50 mL), and the reaction mixture was stirred for 1 h. The solvent was evaporated off, the residue was dissolved in 10% aq. citric acid, and pH was adjusted to 4. After the solution was extracted with EtOAc and the organic layer was dried over Na₂SO₄ and evaporated, N-({4,7,10-tris[(benzyloxy)carbonyl]-1,4,7,10-tetraazacyclododecan-1-yl}acetyl)glycine (5c) was obtained as an amorphous solid. ¹H NMR (300 MHz, CDCl₃): δ 7.29 (m, 15H), 7.00 (s, 1H), 5.28 (s, 6H), 3.90 (br s, 2H), 3.40-3.15 (br m, 14H), 2.80 (br s, 4H). To the stirred solution of 5c (1.5 g, 2.2 mmol) in CH₃CN (100 mL) were added 1b (1.5 g, 2.4 mmol) and DIEA (1.1 mL, 4.3 mmol). To the reaction mixture was added HBTU (0.90 g, 2.4 mmol) and the mixture was stirred for 2 h. The solution was evaporated and the residue was dissolved in EtOAc (100 mL). The solution was washed with 5% aq. citric acid (50 mL×2), 5% aq. Na₂CO₃ (50 mL×2), and brine (50 mL×2), and dried over Na₂SO₄. The solvent was evaporated and flash chromatography afforded tert-butyl N-({4,7,10-tris[(benzyloxy)carbonyl]-1,4,7,10-tetra-azacyclododecan-1-yl} acetyl)glycyl-N-2-{[(9H-fluoren-9-ylmethoxy)carbonyl]amino} ethyl)glycinate (5d) as an amorphous solid. R_{f} 0.3 (CH₃OH/MC 1:19); ¹H NMR (300 MHz, CDCl₃): δ 7.75 (m, 2H), 7.59 (m, 2H), 7.40-7.16 (m, 19H), 5.05-4.85 (br s, 6H), 4.37 (m, 2H), 4.22-4.16 (m, 1H), 3.95 (s, 2H), 3.70-3.32 (br m, 18H), 3.04 (br s, 4H), 1.47 (m, 9H). To the solution of 5d (1.5 g, 1.5 mmol) in MC (25 mL) was added TFA (25 mL). The reaction mixture was stirred for 5 h, the solvent was evaporated off, and flash chromatography afforded 5 as an amorphous solid. R_{f} 0.4 (CH₃OH/MC 1:9); ¹H NMR (300 MHz, CDCl₃): δ 7.72 (m, 2H), 7.57 (m, 2H), 7.40-7.16 (m, 19H), 5.05-4.85 (br s, 6H), 4.37 (m, 1H), 4.20-4.18 (m, 2H), 4.06-3.95 (br s, 4H), 3.70 (br s, 2H), 3.40-3.10 (br m, 18H), 2.83-2.78 (br s, 4H); HRMS exact mass 1013.1403 (M+H)⁺ , calcd for C₅₅H₆₂N₇O₁₂ 1013.1370.

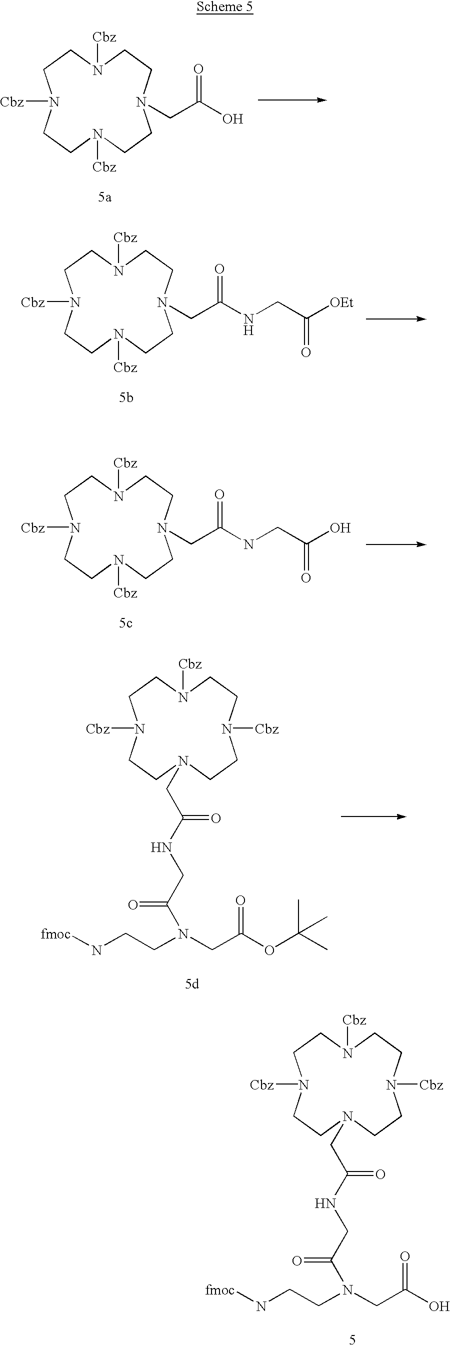

Compound IV was synthesized by using 5 according to the method described in Example 1: MS (MALDI-TOF) m/z 2879.63 (M+H)⁺ (C₁₁₇H₁₆₅N₆₈O₂₆S₂ calcd. 2877.75). Results of Example 2 disclosed that the Lys residue of I is not essential to recognition of Mb. Thus, the PNA 9-mer portion of I is the recognition site. To test whether the PNA 9-mer with Cyc attached at the carboxyl terminus instead of the amino terminus is also useful for the Mb-cleaving catalyst, IV was synthesized.

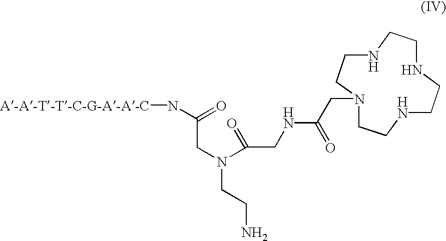

Complexation of Cu(II) ion to IV and kinetic measurement for degradation of Mb were carried out as described in Example 1. When Mb (7.9,M) was incubated with Cu(II)IV (6.4 μM) at pH 8.0 (50 mM Hepes) and 37° C., Mb was degraded with k₀ of 2.2×10⁻³h⁻¹.

### Example 5

{4,10-Bis[(benzyloxy)carbonyl]-1-oxa-4,7,10-triazacyclododecan-7-yl} acetic acid (6) was synthesized according to Scheme 6. tert-Butyl N,N-bis(2-{[(2-nitrophenyl)-sulfonyl] amino}ethyl)glycinate (6a) was synthesized according to the literature (Sasugue, J. M., Segat-Dioury, F.; Sylvestre, I.; Favre-Reguillon, A.; Foos, J.; Madic, C.; Guy, A. Tetrahedron, 2001, 57, 4713). The solution of bromoethyl ether (1 mL, 7.9 mmol) in DMF (100 mL) was added dropwise to the stirred suspension of 6a and anhydrous Na₂CO₃ (3.0 g, 29 mmol) in DMF (100 mL) at 100° C. The reaction mixture was heated overnight and concentrated. The residue was taken up in EtOAc (100 mL). The organic phase was washed with brine (100 mL×2), dried over Na₂SO₄, and concentrated. Flash chromatography afforded tert-butyl {4,10-bis[(2-nitrophenyl)sulfonyl]-1-oxo-4,7,10-tri-azacyclododecan-7-yl}acetate (6b) as an amorphous solid. R_{f} 0.3 (EtOAc/hexane 2:1); ¹H NMR (300 MHz, CDCl₃): δ 8.01-7.98 (m, 2H), 7.69 (m, 4H), 7.60 (m, 2H), 3.68 (m, 4H), 3.55 (m, 4H), 3.72-3.38 (m, 4H), 3.33 (s, 2H), 3.05 (m, 4H), 1.45 (s, 9H). Na₂CO₃ (2.3 g, 22 mmol) was added to the solution of 6b (1.8 g, 2.7 mmol) and thiophenol (0.70 mL, 6.8 mmol) in DMF (30 mL). The reaction mixture was stirred overnight and then concentrated. The residue was dissolved in 10% aq. citric acid and pH was adjusted to 3. The aqueous phase was extracted with EtOAc (100 mL×3). After pH of the aqueous phase was raised to about 13 by adding 1 N aq. NaOH, the aqueous phase was extracted with MC (100 mL×3) and the organic layer was dried over Na₂SO₄ and concentrated. This crude compound (tert-butyl 1-oxa-4,7,10-triazacyclododecan-7-ylacetate (6c)) was used in the next step without further purification. To the solution of 6c (0.50 g, 1.7 mmol) in chloroform (70 mL) was added TEA (0.61 mL, 4.3 mmol). To the stirred solution, benzyl chloroformate (0.43 mL, 3.9 mmol) was added slowly. The reaction mixture was stirred for 3 h, then washed with 5% aq. citric acid (50 mL×3) and concentrated. Flash chromatography afforded dibenzyl 7-(2-tert-butoxy-2-oxo-ethyl)-1-oxa-4,7,10-triazacyclo-dodecane-4,10-dicarbonate (6d) as an oil. R_{f} 0.4 (CH₃OH/MC 1:15); ¹H NMR (300 MHz, CDCl₃): δ 7.32-7.27 (m, 10H), 5.12 (s, 4H), 3.59-3.33 (br m, 14H), 2.99-2.93 (br m, 4H), 1. 45 (s, 9H). To the solution of 6d (0.50 g, 1.0 mmol) in MC (15 mL) was added TFA (10 mL). The reaction mixture was stirred for 5 h. After removal of solvent by evaporation, the residue was dissolved in 10% aq. citric acid and extracted with EtOAc (50 mL×3). The organic layer was washed with brine (50 mL×3), dried over Na₂SO₄, and evaporated to afford 6 as an oil. R_{f} 0.2 (CH₃OH/MC 1:10); ¹H NMR (300 MHz, CDCl₃): δ 7.34-7.25 (m, 10H), 5.11 (m, 4H), 4.23-4.08 (br m, 4H), 3.84-3.50 (br m, 10H), 3,25-3.15 (br m, 4H); MS (MALDI-TOF) m/z 500.50 (M+H)⁺ (C₂₆H₃₄N₃O₇ calcd. 500.58).

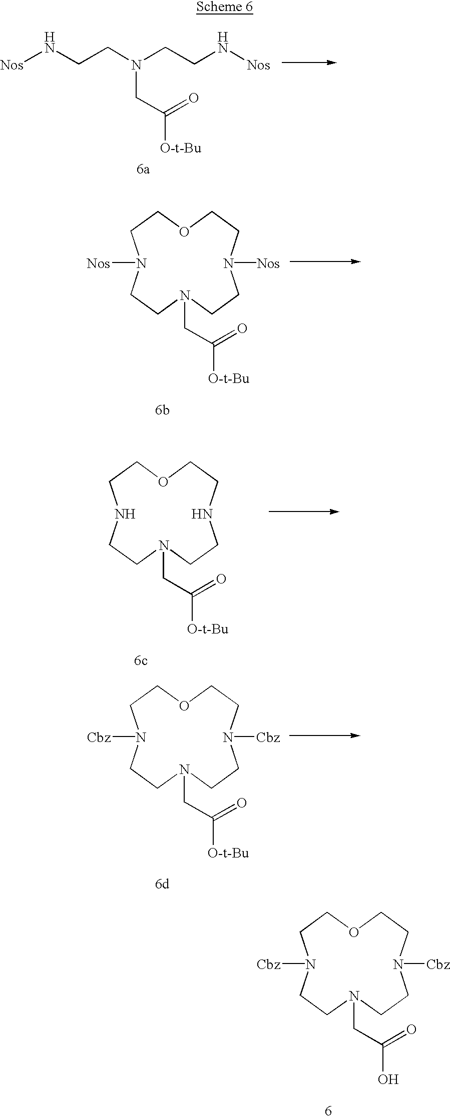

Compound V was synthesized by using 6 according to the method described in Example 1: MS (MALDI-TOF) m/z 2851.49 (M+H)⁺ (C₁₁₁H₁₅₂N₆₃O₂₆S₂ calcd. 2850.75).

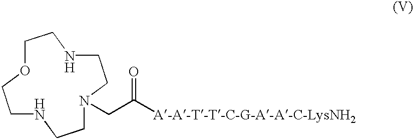

The Co(III) complex of V was obtained as described in Example 1. When Mb (7.9 μM) was incubated with Co(III)V (4.8 μM) at pH 8.0 (50 mM Hepes) or 9.0 (50 mM tris(hydroxymethyl)aminomethane) and 37° C., Mb was degraded with k_{O} of 1.5×10⁻³h⁻¹ or 5.3×10⁻³ h^{-1,} respectively.

### Example 6

{ 4,7-Bis[(benzyloxy)carbonyl]-1,4,7-triazanonan-1}-yl acetic acid (7) was synthesized according to Scheme 7. 1,4,7-Triazanonan-1-ylacetic acid (7a) was synthesized according to the literature (Schulz, D.; Weyhermüiller, T.; Wieghardt, K.; Nuber, B. Inorg. Chim. Acta 1995, 240, 217). To the solution of 7a (3.0 g, 7.4 mmol) in the mixture of aq. NaOH (1 N, 50 mL) and 1,4-dioxane (50 mL) was slowly added benzyl chloroformate (3.0 mL, 22 mmol), and the solution was stirred for 3 h. The solvent was evaporated off, the residue was dissolved in 1 N HCl, and pH was adjusted to 3. After the solution was extracted with EtOAc, the organic layer was dried over Na₂SO₄ and evaporated. By flash chromatography, 7 was obtained as an amorphous solid. R_{f} 0.4 (CH₃OH/MC 1:9); ¹H NMR (300 MHz, CDCl₃): δ 7.34 (m, 10H), 5.15 (d, 4H), 3.38 (m, 10H), 2.74 (br s, 4H), MS (MALDI-TOF) m/z 456.06 (M+H)⁺ (C₂₄H₃₀N₃O₆ calcd. 456.52).

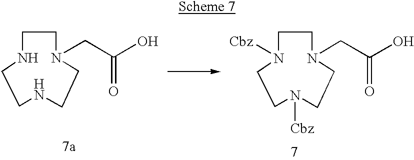

Compound VI was synthesized by using 7 according to the method described in Example 1: MS (MALDI-TOF) m/z 2807.51 (M+H)⁺ (C₁₀₉H₁₄₈N₆₃O₂₅S₂ calcd. 2806.69).

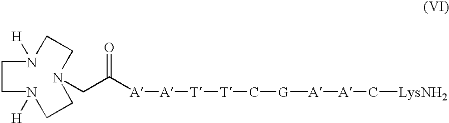

Complexation of Cu(II) ion to VI and kinetic measurement for degradation of Mb were carried out as described in Example 1. When Mb (7.9 μM) was incubated with Cu(II)VI (6.4 μM) at 8.0 (50 mM Hepes) and 37° C., Mb was degraded with k_{O} of 3.6×10⁻³ h⁻¹.

### Example 7

5-[(3 aS,4S,6aR)-2-Oxohexahydro-1H-thieno[3,4-d]imidazol-4-yl]-N-[3-(1,4,7,10-tetraazacyclododecan-1-yl)propyl]pentanamide (VII), a derivative of d-biotin containing cyclen, was synthesized according to Scheme 8. To MC (30 mL) cooled to −60° C. were added oxalyl chloride (1.4 mL, 16 mmol), DMSO (0.89 mL, 13 mmol), the solution of 8a (2.2 g, 10 mmol) in 20 mL MC, and TEA (8.7 mL, 63 mmol) in sequence dropwise. One hour later, the reaction mixture was washed with 50 mM citric acid, dried over Na₂SO₄, and concentrated. Column chromatography on silica gel (EtOAc/hexane 1:1) afforded benzyl 3-oxopropylcarbamate (8b) as a colorless oil. To the solution of 3a (2.3 g, 4.8 mmol) in 20 mL THF were added the solution of 8b (1.0 g, 4.8 mmol) in 40 mL THF and NaBH(OAc)₃ (1.3 g, 6.3 mmol). The reaction mixture was stirred for 1 hr at room temperature. THF was evaporated and the reaction mixture was mixed with 50 mL 0.1 M Na₂CO₃ and extracted with EtOAc (50 mL×2). The collected organic phase was washed with brine, dried over Na₂SO₄, and concentrated. Column chromatography on silica gel (EtOAc/hexane 1:1) afforded tri-tert-butyl 10-(3 {[(benzyloxy)carbonyl]amino}propyl)-1,4,7,10-tetraazacyclo-dodecane-1,4,7-tricarboxylate (8c). ¹H NMR (CDCl₃, 300 MHz): δ 7.32 (m, 5H), 5.09 (s, 2H), 3.56-3.16 (br, 14H), 2.58 (br, 6H), 1.66 (m, 6H), 1.44 (m, 27H). A suspension of 8c (1.0 g, 1.5 mmol) and 500 mg of 10% Pd/C in 100 mL of EtOAc was stirred under 1 atm of H₂ for 24 hr. The catalyst was filtered off on Celite, and the solvent was evaporated off to afford tri-tert-butyl 10-(3-aminopropyl)-1,4,7,10-tetraazacyclododecane-1,4,7-tri-carboxylate (8d) as a colorless oil. ¹H NMR (CDCl₃, 300 MHz): δ 3.56-3.30 (br, 12H), b2.72-2.58 (br, 8H), 1.59 (m, 2H), 1.43 (m, 27H). To the solution of d-biotin (0.22 g, 0.89 mmol) in DMF (5 mL) cooled to 0° C. were added HBTU (0.44 g, 1.2 mmol), 8d (0.47 g, 0.89 mmol) dissolved in DMF (5 mL), and DIEA (200,μl, 1.2 mmol). The reaction mixture was stirred for 6 h at room temperature. The reaction mixture was mixed with 30 mL MC. The mixture was washed with 50 mM citric acid (30 mL×2) and brine, dried over Na,SO₄, and concentrated. Column chromatography on silica gel (CH₃OH/MC 1:9) afforded tri-tert-butyl 10-[3-({5-[(3aS,4S,6aR)-2-oxohexahydro-1H-thieno[3,4-d]imidazol-4-yl]-pentanoyl}amino)propyl]-1,4,7,10-tetraazacyclododecane-1,4,7-tricarboxylate (8e). ′H NMR (CDCl₃, 300 MHz): δ 6.52 (s, 1H), 5.94 (s, 1H), 4.50 (q, 1H), 4.31 (t, 1H), 3.53-3.33 (br, 12H), 3.19 (m, 3H), 2.90 (m, 2H), 2.60 (br, 6H), 2.25 (t, 2H), 1.70 (m, 6H), 1.44 (d, 27H). To the solution of 10% TFA in MC was added 8e (0.51 g, 0.67 mmol) and the reaction mixture was stirred for 3 hr. Ethyl ether was poured to the reaction mixture. White precipitates were collected and dissolved in CH₃OH-diethyl ether mixture. HCl solution was added dropwise to produce the HCl salt of VII. The salt was recrystallized from CH₃OH-diethyl ether. ¹H NMR (CDCl_{3 , 300} MHz): δ 4.57 (t, 1H), 3.85 (q, 1H), 3.30-2.94 (br, 20H), 2.74 (br, 3H), 2.23 (t, 2H), 1.76-1.55 (m, 6H), 1.38 (m, 2H); MS (MALDI-TOF) m/z 456.56 (M+H)⁺ (C₂₁H₄₂N₇O₂S calcd. 456.68).

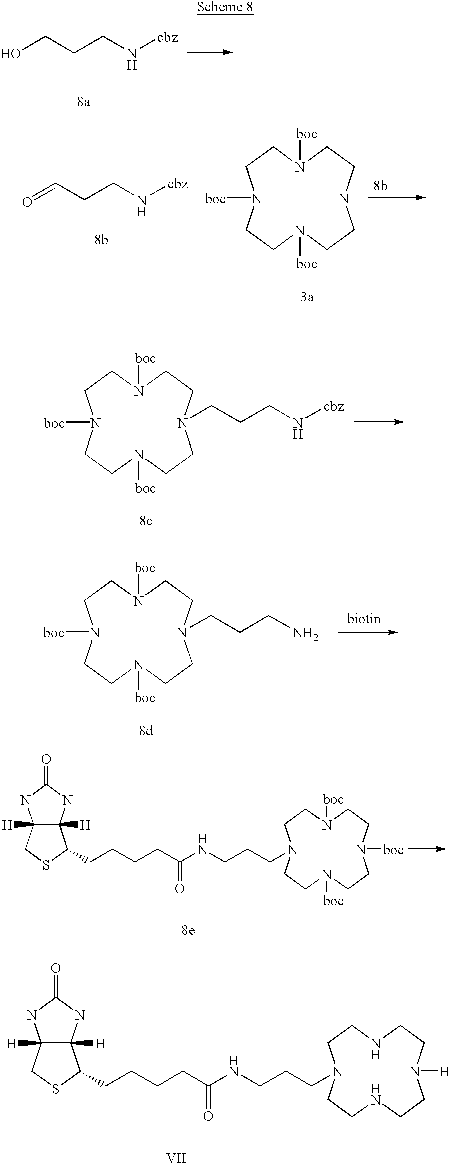

Binding of Cu(II) at the Cyc moiety to produce the Cu(II) complex of VII (Cu(II)VII) was confirmed by UV spectral changes accompanying addition of Cu(II) ion to VII. In view of the strong affinity of biotin for avidin, Cu(II)VII was tested as the protein-cleaving agent for avidin. Complexation of Cu(II)VII to avidin was confirmed by the gel-permeation chromatographic analysis of Cu(II)VII in the presence and absence of avidin. Urea-SDS-PAGE electrophoresis performed on the reaction mixture obtained by incubation of avidin (2.5×10⁻⁵M) with Cu(II)VII (2.5×10⁻⁵M ) under argon for 7 days at pH 6 (50 mM Mes) at 50° C. indicated that about 50% of avidin was cleaved to form smaller fragments. MALDI-TOF MS analysis of the same product indicated that avidin (M.W. 15630) was cleaved into two proteins with M.W. of 10759 and 4922. Examination of the amino acid sequence of avidin and the three-dimensional X-ray crystallographic structure of avid-biotin complex suggested that the cleavage site was Thr35-Ala36 which would produce fragments with M.W. of 10726 and 4922.

### INDUSTRIAL APPLICABILITY

As explained above, the synthetic catalyst designed by the present inventors is composed of the recognition site having affinity for the target protein and the reaction site having activity for cleavage of peptide bond, and so has both the ability to selectively recognize a target protein and the ability to rapidly cleave the peptide bond. Therefore, by using such a synthetic catalyst, it is possible to inhibit the biological activity of the target protein through a selective cleavage thereof under the situation that various proteins are mixed.

1. A synthetic catalyst represented by the following formula (A) which has an ability to selectively cleave a target protein:
(R)(Z)ₙ (A)
in which
n denotes an integer of 1 or more,
R represents a material capable of selectively recognizing and binding a target protein, and
Z represents a metal ion-ligand complex.

2. The synthetic catalyst of claim 1 wherein the ligand is cyclic or acyclic and one to four of the metal-coordinating atoms contained in the ligand are nitrogen atoms.

3. The synthetic catalyst of claim 1 or 2 wherein the skeleton of the ligand is one or more selected from the group consisting of:

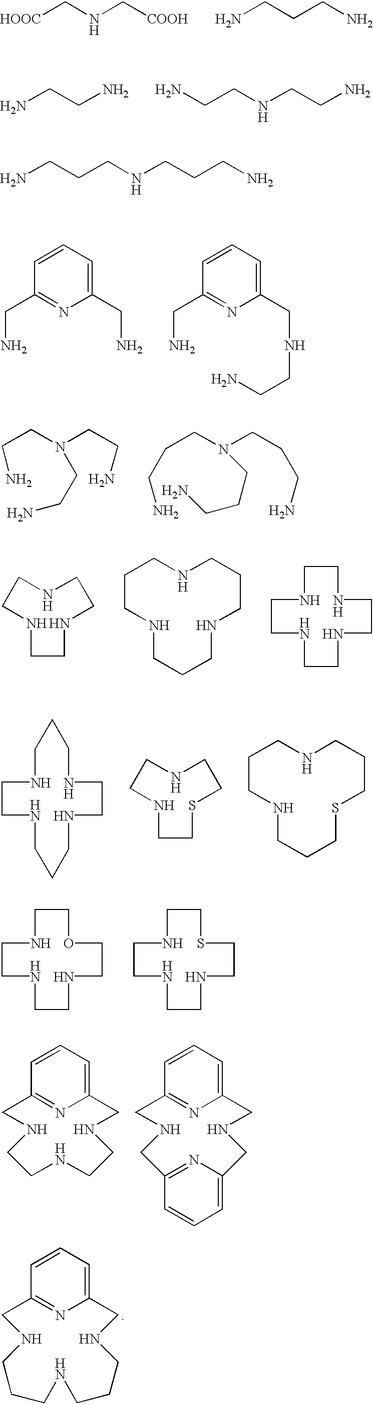

4. The synthetic catalyst of claim 1 wherein the metal ion is one or more selected from the group consisting of Ni(II), Cu(II), Zn(II), Pd(II), Cr(III), Fe(III), Co(III), Rh(III), Ir(III), Ru(III), Pt(IV), Zr(IV), and Hf(IV).

5. The synthetic catalyst of claim 1 wherein R and Z are linked together through a linker.

6. The synthetic catalyst of claim 5 wherein the linker contains a main chain, which has a backbone made of 1 to 30 atoms of boron, carbon, nitrogen, oxygen, silicon, phosphorus, and/or sulfur, belonging to functional groups such as alkyl, aryl, carbonyl, amine, ether, hydroxy, silyl, sulfhydryl, and/or thioether groups as well as derivatives such as amides, imides, esters, and/or thioesters.

7. The synthetic catalyst of claim 5 wherein the linker may contain side chains, each of which has a backbone made of 1 to 30 atoms of boron, carbon, nitrogen, oxygen, silicon, phosphorus, and/or sulfur, belonging to functional groups such as alkyl, aryl, carbonyl, amine, ether, hydroxy, silyl sulfhydryl, and/or thioether groups as well as derivatives such as acids, amides, imides, esters, and/or thioesters.

8. A method for selectively cleaving a target protein characterized by using the synthetic catalyst as defined in claim 1.
